(19) United States
(12) Patent Application Publication
(10) Pub. No.: US 2025/0236580 A1
(43) Pub. Date: Jul. 24, 2025

(54) PROCESS FOR PRODUCING BRANCHED CHAIN FATTY ACIDS AND ESTERS THEREOF

(71) Applicant: INGEVITY SOUTH CAROLINA, LLC, North Charleston, SC (US)

(72) Inventor: Bing Wang, North Charleston, SC (US)

(21) Appl. No.: 18/954,235

(22) Filed: Nov. 20, 2024

Related U.S. Application Data
(60) Provisional application No. 63/622,345, filed on Jan. 18, 2024.

(51) Int. Cl. C07C 51/353; B01D 1/22; B01D 3/10; B01D 5/00; B01D 8/00; B01J 23/755; B01J 25/02; B01J 29/40; B01J 29/65; B01J 29/90; B01J 35/40; B01J 35/54; B01J 35/61; B01J 35/64; B01J 38/02; C07C 51/44; C07C 67/293; C07C 67/54

(52) U.S. Cl. CPC C07C 51/353; B01D 1/225; B01D 3/10; B01D 5/006; B01D 8/00; B01J 23/755; B01J 25/02; B01J 29/40; B01J 29/65; B01J 29/90; B01J 35/40; B01J 35/54; B01J 35/615; B01J 35/643; B01J 38/02; C07C 51/44; C07C 67/293; C07C 67/54

(57) ABSTRACT

The present disclosure describes a method of producing a branched fatty acid or alkyl esters thereof. The method includes subjecting an oleic acid composition comprising at least about 30% of at least one of oleic acid, linoleic acid, or a combination thereof (e.g., at least about 30% oleic acid), to an isomerization reaction in the presence of water and at least one protonated zeolite isomerization catalyst to produce branched unsaturated fatty acids or alkyl esters thereof.

### CROSS-REFERENCE TO RELATED APPLICATIONS

The present applications claims priority to, and the benefit of, U.S. Provisional Application No. 63/622,345, filed 18 Jan. 2024 and titled PROCESS FOR PRODUCING BRANCHED CHAIN FATTY ACIDS AND ESTERS THEREOF, which is incorporated by reference herein in its entirety for all purposes.

### TECHNICAL FIELD

The present disclosure relates to a method of producing a branched fatty acid or alkyl esters thereof.

### BACKGROUND

Branched fatty acids and esters thereof have been commercially available for many years produced from petroleum and biobased sources. Overall, the petroleum-based process of making isostearic acid is tedious and costly. In addition, with recent global focus on sustainability, there is renewed interest in making isostearic acid from biobased materials, which utilizes plant oils and animal fats as feedstocks to create branched fatty acids.

Briefly, the petroleum-based process of producing branched fatty acids involves a mixture of linear alpha-olefins (LAOs) with the desired carbon chain lengths (typically, C16 and C18 linear alpha olefins). These linear alpha-olefins are obtained from various sources, including, e.g., ethylene oligomerization, Fischer-Tropsch synthesis, or natural sources like fats and oils. This is followed by an isomerization step, which introduces branches to the carbon chains. This is done via a catalytic process using a metal complex catalyst (such as zeolites, metal oxides, or organometallic compounds). Then, a hydroformylation step introduces an aldehyde group at the desired position of the molecule, paving the way for the formation of a fatty acid functional group. This step involves a reaction with carbon monoxide and hydrogen in the presence of a rhodium or cobalt catalyst. Alternatively, the carboxylic acid group can be introduced via olefin metathesis. The final step is hydrogenation, which convert unsaturated bonds to saturated isostearic acid. Depending on the quality of the product, a final purification step may be required remove any impurities and achieve the desired quality specifications.

The biobased process of making isosteric acid often involves catalytic transformations like isomerization and hydrogenation. For decades, commercial oleochemical-based isostearic acid have been produced as by-product from dimer acid production using unsaturated fatty acids. In this process, clay is generally used to catalyze the production of a dimer acid. In the process, branches would form on the monomers. The branched monomers are distilled out before hydrogenation to make isostearic acids. This process described in U.S. Pat. No. 2,793,219 A usually produces less than 30% branched fatty acids and over 60% dimers.

Several groups have tried to improve the clay catalyzed process. For example, zeolite catalysts offer significant advantages over clay catalysts in the production of isostearic acid, including higher selectivity, improved activity, enhanced stability, and better control over branching patterns. See, e.g., U.S. Pat. No. 8,748,641 B2 (Ngo et al., a zeolite and a Lewis base are utilized as co-catalyst at 240-280° C., and the protonated ferrierite is converted from the potassium salt of ferrierite with a HCl acid treatment at low temperature), U.S. Pat. No. 10,087,132 B2 (Sarker et al. adds more detail to the reaction of Ngo et al. (4-24 hours) and utilizes high temperature treated zeolite from NH4 salt of ferrierite to produce protonated ferrierite as catalyst), EP 2 702 127 B1 (Bergen-Brenkman et al., a zeolite and a Lewis base are utilized as co-catalysts for making monobranched fatty acids), U.S. Pat. No. 5,677,473 A (Tomifuji et al., process utilizes water and 40-100% oleic acid, under a pressure of 2-50 Kgf/cm² and at a temperature of 150-350° C.), each of which utilizes zeolites to catalyze the branching process instead of clay. These zeolite catalyzed processes, however, have not been commercialized.

While these publications illustrate that proper zeolites can enhance branching formation and that additives (such as triphenylphosphine (TPP)) can suppress dimer formation, these processes are not practical and not sustainable for scaling up. That is why zeolite-based isostearic acid production is not currently being practiced at commercial scale. The additive (such as triphenylphosphine (TPP)) is a bulky Lewis base that was postulated to bind to the Brønsted acid sites at the external surface of the zeolite, but is too large to enter the pores of the zeolite. As result, the external Brønsted acid sites are neutralized, while the internal ones are still available for catalysis. The goal is to counteract the formation of bulky side products, such as dimers/trimers, while the formation of branched fatty acids remains possible inside the micropores of the zeolite.

Thus, there is a need for a process to produce branched fatty acids at temperatures and pressures suitable for commercial scale plants with short reaction times and permit zeolite to be reused easily. The present disclosure provides a process for producing branched chain fatty acids that is both sustainable and commercially viable. The process permits the use of lower grade oleic acid, the use of nickel catalyst, which is a lower cost alternative to traditional catalysts, less zeolite catalyst, lower pressure thereby reducing dimer formation, higher temperature (e.g., about 280° C.) to reduce cycle time, and the ability to not use a Lewis base or oligomerization inhibitors. The process can further include a pellet catalyst to facilitate a fixed bed catalyzed reaction.

### SUMMARY

Presently described are methods of producing branched fatty acid or alkyl esters.

An aspect of the present disclosure provides a method of producing a branched fatty acid or alkyl esters thereof, the method comprising: subjecting an oleic acid composition comprising at least about 30% of at least one of oleic acid, linoleic acid, or a combination thereof (e.g., at least about 30% oleic acid or at least about 60% oleic acid), to an isomerization reaction in the presence of water and at least one protonated zeolite isomerization catalyst to produce branched unsaturated fatty acids or alkyl esters thereof.

In any aspect or embodiment described herein, each of the at least one protonated zeolite isomerization catalyst independently has a ferrierite structure (e.g., HSZ®-722HOA;TOSOH Corporation; Grove City, Ohio) or a Zeolite Socony Mobil-5 (ZSM-5) structure (e.g., HSZ®-822HOA; TOSOH Corporation; Grove City, Ohio).

In any aspect or embodiment described herein, the at least one protonated zeolite isomerization catalyst includes at least one of: (i) a pore size of about 4.0 to about 9.0 angstroms (e.g., about 4.4 to about 6.2 angstroms, about 4.6 to about 6.0 angstroms, or about 4.8 to 5.8 angstroms); (ii) a Brunauer, Emmett and Teller (BET) surface area of about 150 to about 400 m²/g (e.g., about 150 to about 360 or about 170 to about 330 m²/g); (iii) a mole ratio of SiO₂ to Al₂O₃ of about 15 to about 27 (e.g., about 18 to about 24); (iv) a particle size of about 3 to about 22 μm (e.g., about 5 to about 20 μm, about 6 to about 20 μm, about 5 μm, about 6 μm, or about 20 μm); (v) a crystal size of up to about 1.2 μm (e.g., up to about 1.1 μm, up to about 1.0 μm, about ≤0.2 um by about ≤0.6 μm or about 0.1 μm by about 0.5 μm); (vi) a clay binder or an alumina binder; or (vii) a combination thereof.

In any aspect or embodiment described herein, at least one of (i) the oleic acid composition comprises less than about 90% of at least one of oleic acid, linoleic acid, or a combination thereof (e.g., less than about 90% oleic acid); (ii) the oleic acid composition is present in an amount of greater than or equal to about 85.0 weight percent (wt %) (e.g., greater than or equal to about 90.0 wt %, about 85.0 weight percent (wt %) to about 95.0 wt % or about 90 wt % to about 95 wt %) of the isomerization reaction; (iii) the water is present in an amount of no greater than about 4.0 wt % (e.g., about 0.5 wt % to about 4.0 wt % or about 0.5 wt % to about 3.0 wt %) of the isomerization reaction; (iv) the at least one protonated zeolite isomerization catalyst is present in an amount of less than about 5.0 wt % (e.g., less than about 4.0wt %, less than about 3.0 wt %, less than about 2.5 wt %, less than about 2.0 wt %, less than about 0.6 wt %, less than or equal to about 1.5 wt %, about 0.5 to about 5.0 wt %, about 0.5 to about 3.0 wt %, about 0.5 wt % to about 2.5 wt %, about 0.5 wt % to about 2.0 wt %, about 0.5 wt % to about 1.5 wt %, about 2.0 wt % to 5.0 wt %, about 2.0 wt % to 4.5 wt %, about 2.0 wt % to 4.0 wt %, or about 2.5 wt % to 4.0 wt %) of the isomerization reaction; or (v) a combination thereof.

In any aspect or embodiment described herein, at least one of (i) the isomerization reaction does not include a Lew base or an oligomerization inhibitor; (ii) the isomerization reaction is performed for up to about 30 hours (e.g., up to about 26 hours, up to about 20 hours, up to about 15 hours, up to about 10 hours, up to about 6 hours, up to about 4 hours, about 1 to about 30 hours, about 1 to about 26 hours, about 1 to about 20 hours, about 1 to about 15 hours, about 1 to about 10 hours, about 1 to about 6 hours, about 1 hour to about 4 hours, about 2 hours to about 6 hours, or about 2 hours to about 4 hours); or (iii) a combination thereof.

In any aspect or embodiment described herein, at least one of (i) the isomerization reaction does not include a Lewis based or an oligomerization inhibitor; (ii) the isomerization reaction is performed for up to about 10 hours (e.g., up to about 6 hours, up to about 4 hours, about 1 to about 10 hours, about 1 to about 6 hours, about 1 hour to about 4 hours, about 2 hours to about 6 hours, or about 2 hours to about 4 hours); or (iii) a combination thereof.

In any aspect or embodiment described herein, the isomerization reaction is performed for about 24 to about 48 hours (e.g., about 24 to about 42 hours, about 24 to about 36 hours, about 1 hour to about 4 hours, about 2 hours to about 6 hours, or about 2 hours to about 4 hours).

In any aspect or embodiment described herein, the isomerization reaction was performed (i) at about 200° C. to about 300° C. (e.g., about 240° C. to about 280° C., about 240° C. to about 270° C., or about 260° C.), (ii) with a final pressure of about 50 pounds per square inch to about 500 pounds per square inch (e.g., about 100 to about 400 or about 100 to about 350 pounds per square inch), (iii) or a combination thereof of (i) and (ii).

In any aspect or embodiment described herein, wherein the isomerization reaction was performed with a final pressure of less than or equal to about 125 pounds per square inch (e.g., less than or equal to about 120 pounds per square inch, about 100 to about 125 pounds per square inch, or about 100 to about 120 pounds per square inch).

In any aspect or embodiment described herein, at least one of (i) the method produces less than or equal to about 40% dimers (e.g., less than or equal to about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, or about 10% dimers); (ii) the method provides at least about 90% conversion (e.g., at least about 91%, about 92%, about 93%, about 94%, about 95%, about 96%, or about 97% conversion); or (iii) a combination thereof.

In any aspect or embodiment described herein, the method further comprises, consists essentially of, or consists of, recovering or isolating the at least one protonated zeolite isomerization catalyst, and optionally regenerating the at least one protonated zeolite isomerization catalyst.

In any aspect or embodiment described herein, the at least one protonated zeolite isomerization catalyst is in a pellet form.

In any aspect or embodiment described herein, the method further comprises, consists essentially of, or consists of, regenerating the at least one protonated zeolite isomerization catalyst (e.g., the at least one protonated zeolite isomerization catalyst is in a pellet form) by heating (e.g., heating to a temperature of about 300° C. to about 500° C. for at least 2 hours, such as about 2 hours to about 5 hours) the at least one protonated zeolite isomerization catalyst.

In any aspect or embodiment described herein, the method further comprises, consists essentially of, or consists of, regenerating the at least one protonated zeolite isomerization catalyst (e.g., the at least one protonated zeolite isomerization catalyst is in a pellet form) by heating (e.g., heating to a temperature of about 100° C. to about 300° C. for at least 2 hours, such as about 2 hours to about 5 hours) the at least one protonated zeolite isomerization catalyst.

In any aspect or embodiment described herein, the at least one protonated zeolite isomerization catalyst (e.g., the at least one protonated zeolite isomerization catalyst is in a pellet form) is located in a fixed bed of a reactor, and the method optionally further comprises, consists essentially of, or consists of, removing the at least one protonated zeolite isomerization catalyst from the fixed bed prior to heating.

In any aspect or embodiment described herein, recovering or isolating the at least one protonated zeolite isomerization catalyst comprises filtering the branched unsaturated fatty acids or alkyl esters to obtain a filtrate comprising filtered branched unsaturated fatty acids or alkyl esters and a particulate comprising the at least one protonated zeolite isomerization catalyst, and optionally, regenerating the at least one protonated zeolite isomerization catalyst through at least one of an acid treatment (e.g., an acid treatment comprising an acid solution and a solvent, such as a polar solvent or a non-polar solvent), a heat treatment (e.g., 100° C. to about 500° C. for about 2 hours to about 5 hours), or a combination thereof, of the particulate.

In any aspect or embodiment described herein, the method further comprises, consists essentially of, or consists of, subjecting a reaction product of the isomerization reaction or the branched unsaturated fatty acids or alkyl esters thereof to an additional isomerization reaction in the presence of water and (i) at least one protonated zeolite isomerization catalyst or (ii) a clay catalyst to produce branched unsaturated fatty acids or alkyl esters thereof.

In any aspect or embodiment described herein, subjecting the reaction product of the isomerization reaction or the branched unsaturated fatty acids or alkyl esters thereof to the additional isomerization reaction comprises adding the water and (i) the at least one protonated zeolite isomerization catalyst or (ii) the clay catalyst to the isomerization reaction or the branched unsaturated fatty acids or alkyl esters thereof.

In any aspect or embodiment described herein, at least one of (i) the additional isomerization reaction does not include a Lew base or an oligomerization inhibitor; (ii) the additional isomerization reaction is performed for up to about 15 hours (e.g., up to about 12 hours, up to about 10 hours, about 1 to about 15 hours, about 1 to about 12 hours, about 1 to about 10 hours, about 5 to about 15 hours, about 5 to about 12 hours, about 7 to about 11 hours, or about 8 hour to about 10 hours); (iii) the additional isomerization reaction was performed at about 200° C. to about 300° C. (e.g., about 240° C. to about 280° C., about 240° C. to about 270° C., or about 260° C.); (iv) the additional isomerization reaction was performed with a final pressure of less than or equal to about 125 pounds per square inch (e.g., less than or equal to about 120 pounds per square inch, about 100 to about 125 pounds per square inch, or about 100 to about 120 pounds per square inch); or (v) a combination thereof.

In any aspect or embodiment described herein, the further comprises, consists essentially of, or consists of, hydrogenating the branched unsaturated fatty acids or alkyl esters thereof (e.g., the branched unsaturated fatty acids or alkyl esters isolated through distilling or the filtered branched unsaturated fatty acids or alkyl esters) in the presence of a hydrogenation catalyst to produce branched saturated fatty acids or alkyl esters thereof.

In any aspect or embodiment described herein, the hydrogenation catalyst does not include a palladium-based catalyst.

In any aspect or embodiment described herein, hydrogenating the branched unsaturated fatty acids or alkyl esters thereof comprises at least one of: (i) heating a reaction mixture to about 200° C. to about 230° C. (e.g., about 215° C.); (ii) pressurizing a reaction mixture (e.g., pressurizing the reaction mixture with hydrogen) to about 90 pounds per square inch to about 110 pounds per square inch; (iii) reacting a reaction mixture until the oleic acid content is below about 2% (e.g., below about 1.5%, below about 1.2%, below about 1.0%, below about 0.9%, or below about 0.8%); (iv) reacting a reaction mixture for at least about 5 hours (e.g., about 5 hours to about 18 hours or about 6 hours); or (v) a combination thereof.

In any aspect or embodiment described herein, at least one of (i) the hydrogenation catalyst comprises at least one of a spongy nickel catalyst (e.g., Raney® nickel), a supported nickel catalyst (e.g., a silica-alumina support nickel, an alumina support nickel, silica supported nickel, or a combination thereof), a polymer-supported nickel catalyst, or a combination thereof; (ii) the hydrogenation catalyst is present in an amount of no greater than about 3 wt % (e.g., no greater than about 2 wt % or no greater than about 1 wt %) of the reaction mixture; (iii) the branched unsaturated fatty acids or alkyl esters thereof is present in an amount of at least about 97 wt % (e.g., at least about 98 wt % or at least about 99 wt %) of the reaction mixture; or (iv) a combination thereof.

In any aspect or embodiment described herein, hydrogenating the branched unsaturated fatty acids or alkyl esters thereof comprises preparing a reaction mixture comprising a hydrogenation catalyst and the branched unsaturated fatty acids or alkyl esters thereof (e.g., the filtered branched unsaturated fatty acids or alkyl esters or the branched unsaturated fatty acids or alkyl esters isolated through distilling).

In any aspect or embodiment described herein, the method further comprises, consists essentially of, or consists of, distilling the branched unsaturated fatty acids or alkyl esters.

In any aspect or embodiment described herein, at least one of (i) distilling is wiped film evaporator vacuum distillation; (ii) distilling the branched unsaturated fatty acids or alkyl esters includes: isolating the branched unsaturated fatty acids or alkyl esters, isolating the zeolite isomerization catalyst, isolating at least one of dimer fatty acids, trimer fatty acids, or a combination thereof, or a combination thereof; or (iii) a combination thereof.

In any aspect or embodiment described herein, distilling is wiped film evaporator vacuum distillation, and at least one of: (i) a still body jacket of the wiped film evaporator is heated to a temperature of about 220° C. to about 260° C. (e.g., about 240° C.) with vacuum (e.g., about 4 mmHg to about 8 mmHg or about 6 mmHg); (ii) a condenser of the wiped film evaporator is set to a temperature of about 50° C. to about 70° C. (e.g., about 60° C.); (iii) a cold trap of the wiped film evaporator is set to a temperature of about 3° C. to about 10° C. (e.g., about 5° C.); or (iv) a combination thereof.

An aspect of the present disclosure provides a composition produced according to the method of the present disclosure.

An additional aspect of the present disclosure provides a composition comprising, consisting essentially of, or consisting of: branched unsaturated fatty acids or alkyl esters monomers in an amount of about 30.0 wt % to about 90.0 wt %, based on the total weight of the composition; and branched unsaturated fatty acid or alkyl ester oligomers (e.g., dimers) in an amount of about 10.0 wt % to about 30.0 wt %, based on the total weight of the composition.

Another aspect of the present disclosure provides a composition comprising, consisting essentially of, or consisting of: branched unsaturated fatty acids or alkyl esters monomers in an amount of about 30.0 wt % to about 90.0 wt %, based on the total weight of the composition; and branched unsaturated fatty acid or alkyl ester oligomers (e.g., dimers) in an amount of about 10.0 wt % to about 30.0 wt %, based on the total weight of the composition.

The preceding general areas of utility are given by way of example only and are not intended to be limiting on the scope of the present disclosure and appended claims. Additional objects and advantages associated with the polymers, compositions, methods, and processes of the present disclosure will be appreciated by one of ordinary skill in the art in light of the instant claims, description, and examples. For example, the various aspects and embodiments of the present disclosure can be utilized in numerous combinations, all of which are expressly contemplated by the present disclosure. These additional advantages objects and embodiments are expressly included within the scope of the present disclosure. The publications and other materials used herein to illuminate the background of the invention, and in particular cases, to provide additional details respecting the practice, are incorporated by reference.

### DETAILED DESCRIPTION

There is a need for a process to produce branched fatty acids at temperatures and pressures suitable for commercial scale plants with short reaction times and permit zeolite to be reused easily. The inventors of the present disclosure have surprisingly and unexpected discovered a process for producing branched chain fatty acids that is both sustainable and commercially viable. Surprisingly, the process of the present disclosure permits the use of lower grade oleic acid, less zeolite catalyst for isomerization, lower pressures thereby reducing dimer formation and easier to scale up production, higher temperature (e.g., about 280° C.) to reduce cycle time, and the ability to not use Lewis bases as oligomerization inhibitors. The process can further include a pellet catalyst to facilitate a fixed bed catalyzed reaction and/or a step-wise isomerization in order to increase the rate of conversion.

Thus, the present disclosure provides a method of producing a branched fatty acid or alkyl esters thereof, the method comprising: subjecting an oleic acid composition comprising at least about 60% of at least one of oleic acid, linoleic acid, or a combination thereof (e.g., at least about 60% oleic acid), to an isomerization reaction in the presence of water and at least one protonated zeolite isomerization catalyst to produce branched unsaturated fatty acids or alkyl esters thereof, wherein each of the at least one protonated zeolite isomerization catalyst; and distilling the branched unsaturated fatty acids or alkyl esters.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value in the range, to the tenth of the unit of the lower limit unless the context clearly dictates otherwise (for example, in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

It should also be understood that, in certain methods or processes described herein that include more than one step or act, the order of the steps or acts of the method is not necessarily limited to the order in which the steps or acts of the method are recited unless the context indicates otherwise.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (that is, to at least one or one or more of) of the grammatical object of the article unless the context clearly indicates otherwise. By way of example, “an element” means one element or more than one element, unless otherwise indicated.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, that is, “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (that is, “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended (that is, to mean including but not limited to). It is expressly contemplated that all embodiments, and claims reciting one of the open-ended transitional phrases can be written with any other transitional phrase, which may be more limiting, unless clearly precluded by the context or art. Only the transitional phrases “consisting of” and “consisting essentially of” shall be closed or semi-closed transitional phrases, respectively, as set forth in the United States Patent Office Manual of Patent Examining Procedures, Section 2111.03.

The term “independently” is used herein to indicate that the variable, which is independently applied, varies independently from application to application.

The term “effective” is used to describe an amount of a compound, composition, or component which, when used within the context of its intended use, effects an intended result. The term effective subsumes all other effective amount or effective concentration terms, which are otherwise described or used in the present application.

As discussed herein, the inventors of the present disclosure surprisingly and unexpected discovered a method for producing branched chain fatty acids that is both sustainable and commercially viable. Surprisingly, the method of the present disclosure permits the use of lower grade oleic acid, the use of nickel catalyst for hydrogenation, which is a lower cost alternative to traditional catalysts, less zeolite catalyst for isomerization, lower pressures thereby reducing dimer formation and easier to scale up production, higher temperature (e.g., about 280° C.) to reduce cycle time, and the ability to not use a Lew base, as an oligomerization inhibitor. The process can further include a pellet catalyst to facilitate a fixed bed catalyzed reaction and/or a step-wise isomerization in order to increase the rate of conversion.

An aspect of the present disclosure provides a method of producing a branched fatty acid or alkyl esters thereof, the method comprising: subjecting an oleic acid composition comprising at least about 30% of at least one of oleic acid, linoleic acid, or a combination thereof (e.g., at least about 60% of at least one of oleic acid, linoleic acid, or a combination thereof; at least about 60% oleic acid; or at least 30% oleic acid), to an isomerization reaction in the presence of water and at least one protonated zeolite isomerization catalyst to produce branched unsaturated fatty acids or alkyl esters thereof.

An aspect of the present disclosure provides a method of producing a branched fatty acid or alkyl esters thereof, the method comprising: subjecting an oleic acid composition comprising at least about 60% of at least one of oleic acid, linoleic acid, or a combination thereof (e.g., at least about 60% oleic acid), to an isomerization reaction in the presence of water and at least one protonated zeolite isomerization catalyst to produce branched unsaturated fatty acids or alkyl esters thereof.

A further aspect of the present disclosure provides a composition produced according to the method of the present disclosure.

In any aspect or embodiment described herein, the method produces less than or equal to about 40% dimers. For example, in any aspect or embodiment described herein, the method produces Sabout 39%. ≤about 38%, ≤about 37%. ≤about 36%. ≤about 35%, ≤about 34%, ≤about 33%, ≤about 32%, ≤about 31%, ≤about 30%, ≤about 29%, ≤about 28%, ≤about 27%, ≤about 26%, ≤about 25%, ≤about 24%, ≤about 23%, ≤about 22%, ≤about 21%, ≤about 20%, ≤about 19%, ≤about 18%, ≤about 17%, ≤about 16%, ≤about 15%, ≤about 14%, ≤about 13%, ≤about 12%, ≤about 11%, or ≤about 10% dimers. In any aspect or embodiment described herein, the method provides at least about 90% conversion. For example, in any aspect or embodiment described herein, the method provides > about 91%, >about 92%, >about 93%, >about 94%, >about 95%, >about 96%, >about 97%, >98%, or >99% conversion.

In any aspect or embodiment described herein, the water is present in an amount of no greater than about 4.0 wt % (e.g., about 0.5 wt % to about 4.0 wt % or about 0.5 wt % to about 3.0 wt %) of the isomerization reaction. For example, in any aspect or embodiment described herein, the water is present in an amount of ≤about 4.0 wt %, ≤about 3.5 wt %, ≤about 3.0 wt %, ≤about 2.5 wt %, ≤about 2.0 wt %, ≤about 1.5 wt %, ≤about 1.0 wt %, or ≤about 0.5 wt %, about 0.5 wt % to about 4.0 wt %, about 1.0 wt % to about 4.0 wt %, about 1.5 wt % to about 4.0 wt %, about 2.0 wt % to about 4.0 wt %, about 2.5 wt % to about 4.0 wt %, about 3.0 wt % to about 4.0 wt %, about 0.5 wt % to about 3.5 wt %, about 1.0 wt % to about 3.5 wt %, about 1.5 wt % to about 3.5 wt %, about 2.0 wt % to about 3.5 wt %, about 2.5 wt % to about 3.5 wt %, about 0.5 wt % to about 3.0 wt %, about 1.0 wt % to about 3.0 wt %, about 1.5 wt % to about 3.0 wt %, about 2.0 wt % to about 3.0 wt %, about 0.5 wt % to about 2.5 wt %, about 1.0 wt % to about 2.5 wt %, about 1.5 wt % to about 2.5 wt %, about 0.5 wt % to about 2.0 wt %, about 1.0 wt % to about 2.0 wt %, or about 0.5 wt % to about 1.5 wt %.

In any aspect or embodiment described herein, the method further comprises, consists essentially of, or consists of, subjecting a reaction product of the isomerization reaction or the branched unsaturated fatty acids or alkyl esters thereof to an additional isomerization reaction in the presence of water and (i) at least one protonated zeolite isomerization catalyst or (ii) a clay catalyst to produce branched unsaturated fatty acids or alkyl esters thereof.

In any aspect or embodiment described herein, subjecting the reaction product of the isomerization reaction or the branched unsaturated fatty acids or alkyl esters thereof to the additional isomerization reaction comprises adding the water and (i) the at least one protonated zeolite isomerization catalyst or (ii) the clay catalyst to the isomerization reaction or the branched unsaturated fatty acids or alkyl esters thereof.

### Isomerization Reaction and Additional Isomerization Reaction

In any aspect or embodiment described herein, the isomerization reaction was performed at about 200° C. to about 300° C. (e.g., about 240° C. to about 280° C., about 240° C. to about 270° C. or about 260° C.). For example, in any aspect or embodiment described herein, the isomerization reaction was performed at about 200° C. to about 300° C., about 200° C. to about 280° C., about 200° C. to about 260° C., about 200° C. to about 240° C., about 200° C. to about 220° C., about 220° C. to about 300° C., about 220° C. to about 280° C., about 220° C. to about 260° C., about 220° C. to about 240° C., about 240° C. to about 300° C., about 240° C. to about 280° C., about 240° C. to about 260° C., about 260° C. to about 300° C., about 260° C. to about 280° C., or about 280° C. to about 300° C.

In any aspect or embodiment described herein, the additional isomerization reaction was performed at about 200° C. to about 300° C. (e.g., about 240° C. to about 280° C., about 240° C. to about 270° C., or about 260° C.). For example, in any aspect or embodiment described herein, the additional isomerization reaction was performed at about 200° C. to about 300° C., about 200° C. to about 280° C., about 200° C. to about 260° C., about 200° C. to about 240° C., about 200° C. to about 220° C., about 220° C. to about 300° C., about 220° C. to about 280° C., about 220° C. to about 260° C., about 220° C. to about 240° C., about 240° C. to about 300° C., about 240° C. to about 280° C., about 240° C. to about 260° C., about 260° C. to about 300° C., about 260° C. to about 280° C., or about 280° C. to about 300° C.

In any aspect or embodiment described herein, the isomerization reaction was performed with a final pressure of about 50 pounds per square inch to about 500 pounds per square inch (e.g., about 100 to about 400 or about 100 to about 350 pounds per square inch). For example, in any aspect or embodiment described herein, the isomerization reaction was performed with a final pressure of about 50 to about 500, about 50 to about 450, about 50 to about 400, about 50 to about 350, about 50 to about 300, about 50 to about 250, about 50 to about 200, about 50 to about 150, about 100 to about 500, about 100 to about 450, about 100 to about 400, about 100 to about 350, about 100 to about 300, about 100 to about 250, about 100 to about 200, about 150 to about 500, about 150 to about 450, about 150 to about 400, about 150 to about 350, about 150 to about 300, about 150 to about 250, about 200 to about 500, about 200 to about 450, about 200 to about 400, about 200 to about 350, about 200 to about 300, about 250 to about 500, about 250 to about 450, about 250 to about 400, about 250 to about 350, about 300 to about 500, about 300 to about 450, about 300 to about 400, about 350 to about 500, about 350 to about 450, or about 400 to about 500 pounds per square inch.

In any aspect or embodiment described herein, the additional isomerization reaction was performed with a final pressure of about 50 pounds per square inch to about 500 pounds per square inch (e.g., about 100 to about 400 or about 100 to about 350 pounds per square inch). For example, in any aspect or embodiment described herein, the isomerization reaction was performed with a final pressure of about 50 to about 500, about 50 to about 450, about 50 to about 400, about 50 to about 350, about 50 to about 300, about 50 to about 250, about 50 to about 200, about 50 to about 150, about 100 to about 500, about 100 to about 450, about 100 to about 400, about 100 to about 350, about 100 to about 300, about 100 to about 250, about 100 to about 200, about 150 to about 500, about 150 to about 450, about 150 to about 400, about 150 to about 350, about 150 to about 300, about 150 to about 250, about 200 to about 500, about 200 to about 450, about 200 to about 400, about 200 to about 350, about 200 to about 300, about 250 to about 500, about 250 to about 450, about 250 to about 400, about 250 to about 350, about 300 to about 500, about 300 to about 450, about 300 to about 400, about 350 to about 500, about 350 to about 450, or about 400 to about 500 pounds per square inch.

In any aspect or embodiment described herein, wherein the isomerization reaction was performed with a final pressure of less than or equal to about 125 or about 120 pounds per square inch (e.g., less than or equal to about 120 pounds per square inch, about 100 to about 125 pounds per square inch, or about 100 to about 120 pounds per square inch). For example, in any aspect or embodiment described herein, the isomerization reaction was performed with a final pressure of about 50 to about 125, about 50 to about 120, about 50 to about 110, about 50 to about 100, about 50 to about 90, about 50 to about 80, about 50 to about 70, about 50 to about 60, about 60 to about 125, about 60 to about 120, about 60 to about 110, about 60 to about 100, about 60 to about 90, about 60 to about 80, about 60 to about 70, about 70 to about 125, about 70 to about 120, about 70 to about 110, about 70 to about 100, about 70 to about 90, about 70 to about 80, about 80 to about 125, about 80 to about 120, about 80 to about 110, about 80 to about 100, about 80 to about 90, about 90 to about 125, about 90 to about 120, about 90 to about 110, about 90 to about 100, about 100 to about 125, about 100 to about 120, about 100 to about 110, about 110 to about 125, about 110 to about 120, or about 115 to about 125 pounds per square inch.

In any aspect or embodiment described herein, the additional isomerization reaction was performed with a final pressure of less than or equal to about 125 pounds per square inch (e.g., less than or equal to about 120 pounds per square inch, about 100 to about 125 pounds per square inch, or about 100 to about 120 pounds per square inch). For example, in any aspect or embodiment described herein, the additional isomerization reaction was performed with a final pressure of about 50 to about 125, about 50 to about 120, about 50 to about 110, about 50 to about 100, about 50 to about 90, about 50 to about 80, about 50 to about 70, about 50 to about 60, about 60 to about 125, about 60 to about 120, about 60 to about 110, about 60 to about 100, about 60 to about 90, about 60 to about 80, about 60 to about 70, about 70 to about 125, about 70 to about 120, about 70 to about 110, about 70 to about 100, about 70 to about 90, about 70 to about 80, about 80 to about 125, about 80 to about 120, about 80 to about 110, about 80 to about 100, about 80 to about 90, about 90 to about 125, about 90 to about 120, about 90 to about 110, about 90 to about 100, about 100 to about 125, about 100 to about 120, about 100 to about 110, about 110 to about 125, about 110 to about 120, or about 115 to about 125 pounds per square inch.

### Zeolite Isomerization Catalyst

In any aspect or embodiment described herein, the zeolite is a crystalline, hydrated aluminosilicates that typically have rigid anionic frameworks containing well-defined channels and cavities. In any aspect or embodiment described herein, spent zeolite is a zeolite catalyst that has undergone an activation and one or more (e.g., 1, 2, 3, 4, 5, 6, 7, or more) reaction cycles, and optionally will undergo a subsequent regeneration cycle. In any aspect or embodiment described herein, the method further comprises, consists essentially of, or consists of, recovering or isolating the at least one protonated zeolite isomerization catalyst, and optionally regenerating the at least one protonated zeolite isomerization catalyst. In any aspect or embodiment described herein, regeneration or regenerating is the reactivation (e.g., at least one of a heat treatment, an acid treatment, or a combination thereof) of a spent zeolite to reactivate its catalytic properties after being used for one or more (e.g., 1, 2, 3, 4, 5, 6, 7, or more) reaction cycles for repeated reuse in the reaction process.

In any aspect or embodiment described herein, each of the at least one protonated zeolite isomerization catalyst independently has an A-type structure, an X-type structure, a BETA-type structure, a Zeolite Socony Mobil-5 (ZSM-5)-type structure, a ferrierite-type structure, a mordenite-type structure, a L-type structure, or a Y-type structure. In any aspect or embodiment described herein, each of the at least one protonated zeolite isomerization catalyst independently has a ferrierite structure (e.g., HSZ®-722HOA;TOSOH Corporation; Grove City, Ohio) or a Zeolite Socony Mobil-5 (ZSM-5) structure (e.g., HSZ®-822HOA; TOSOH Corporation; Grove City, Ohio). In any aspect or embodiment described herein, each of the at least one protonated zeolite isomerization catalyst independently has a ferrierite structure (e.g., HSZ®-722HOA;TOSOH Corporation; Grove City, Ohio). In any aspect or embodiment described herein, each of the at least one protonated zeolite isomerization catalyst independently has a Zeolite Socony Mobil-5 (ZSM-5) structure (e.g., HSZ®-822HOA; TOSOH Corporation; Grove City, Ohio).

Further, the at least one protonated zeolite isomerization catalyst used in the present disclosure contains a template (also referred to as a structure directing agent) or a proton (H⁺) counter cation in the crystal structure.

In any aspect or embodiment described herein, the specific surface area (e.g., Brunauer, Emmett and Teller (BET) surface area) of the at least one protonated zeolite isomerization catalyst is about 100 to about 800 m²/g (e.g., about 150 to about 700 m²/g, about 150 to about 600 m²/g, or about 150 to about 500 m²/g). In any aspect or embodiment described herein, the at least one protonated zeolite isomerization catalyst has a Brunauer, Emmett and Teller (BET) surface area of about 150 to about 400 m²/g (e.g., about 150 to about 360 or about 170 to about 330 m²/g). For example, in any aspect or embodiment described herein, the at least one protonated zeolite isomerization catalyst has a Brunauer, Emmett and Teller (BET) surface area of about 150 to about 400 m²/g, about 150 to about 350 m²/g, about 150 to about 300 m²/g, about 150 to about 250 m²/g, about 150 to about 200 m²/g, about 200 to about 400 m²/g, about 200 to about 350 m²/g, about 200 to about 300 m²/g, about 200 to about 250 m²/g, about 250 to about 400 m²/g, about 250 to about 350 m²/g, about 250 to about 300 m²/g, about 300 to about 400 m²/g, about 300 to about 350 m²/g, or about 350 to about 400 m²/g.

In any aspect or embodiment described herein, the pore size/diameter of the at least one protonated zeolite isomerization catalyst is about 2 angstrom to about 10 angstrom (e.g., about 5 angstrom to about 8 angstrom, about 6 angstrom to about 7.5 angstrom, or about 6.5 angstrom to about 7.5 angstrom). In any aspect or embodiment described herein, the at least one protonated zeolite isomerization catalyst has a pore size/diameter of about 4.0 to about 9.0 angstroms (e.g., about 4.4 to about 6.2 angstroms, about 4.6 to about 6.0 angstroms, or about 4.8 to 5.8 angstroms). For example, in any aspect or embodiment described herein, the at least one protonated zeolite isomerization catalyst has a pore size/diameter of about 4.0 to about 9.0 angstroms, about 4.0 to about 8.0 angstroms, about 4.0 to about 7.0 angstroms, about 4.0 to about 6.0 angstroms, about 4.0 to about 5.0 angstroms, about 5.0 to about 9.0 angstroms, about 5.0 to about 8.0 angstroms, about 5.0 to about 7.0 angstroms, about 5.0 to about 6.0 angstroms, about 6.0 to about 9.0 angstroms, about 6.0 to about 8.0 angstroms, about 6.0 to about 7.0 angstroms, about 7.0 to about 9.0 angstroms, about 7.0 to about 8.0 angstroms, or about 8.0 to about 9.0 angstroms.

In any aspect or embodiment described herein, the at least one protonated zeolite isomerization catalyst has a mole ratio of SiO₂ to Al₂O₃ of about 2 to about 1500 (e.g., about 10 to about 1500 or about 15 to about 300). In any aspect or embodiment described herein, the at least one protonated zeolite isomerization catalyst has a mole ratio of SiO₂to Al₂O₃ of about 15 to about 27 (e.g., about 18 to about 24). For example, in any aspect or embodiment described herein, the at least one protonated zeolite isomerization catalyst has a mole ratio of SiO₂to Al₂O₃ of about 15 to about 27, about 15 to about 25, about 15 to about 23, about 15 to about 21, about 15 to about 19, about 15 to about 17, about 17 to about 27, about 17 to about 25, about 17 to about 23, about 17 to about 21, about 17 to about 19, about 19 to about 27, about 19 to about 25, about 19 to about 23, about 19 to about 21, about 21 to about 27, about 21 to about 25, about 21 to about 23, about 23 to about 27, about 23 to about 25, or about 25 to about 27.

In any aspect or embodiment described herein, the at least one protonated zeolite isomerization catalyst has a particle size of about 3 to about 22 μm (e.g., about 5 to about 20 μm, about 6 to about 20 μm, about 5 μm, about 6 μm, or about 20 μm). For example, in any aspect or embodiment described herein, the at least one protonated zeolite isomerization catalyst has a particle size of about 3 to about 22 μm, about 3 to about 20 μm, about 3 to about 18 μm, about 3 to about 16 μm, about 3 to about 14 μm, about 3 to about 12 μm, about 3 to about 10 μm, about 3 to about 8 μm, about 3 to about 6 μm, about 5 to about 22 μm, about 5 to about 20 μm, about 5 to about 18 μm, about 5 to about 16 μm, about 5 to about 14 μm, about 5 to about 12 μm, about 5 to about 10 μm, about 5 to about 8 μm, about 7 to about 22 μm, about 7 to about 20 μm, about 7 to about 18 μm, about 7 to about 16 μm, about 7 to about 14 μm, about 7 to about 12 μm, about 7 to about 10 μm, about 9 to about 22 μm, about 9 to about 20 μm, about 9 to about 18 μm, about 9 to about 16 μm, about 9 to about 14 μm, about 9 to about 12 μm, about 11 to about 22 μm, about 11 to about 20 μm, about 11 to about 18 μm, about 11 to about 16 μm, about 11 to about 14 μm, about 13 to about 22 μm, about 13 to about 20 μm, about 13 to about 18 μm, about 13 to about 16 μm, about 15 to about 22 μm, about 15 to about 20 μm, about 15 to about 18 μm, about 17 to about 22 um, about 17 to about 20 μm, or about 19 to about 22 μm.

In any aspect or embodiment described herein, the at least one protonated zeolite isomerization catalyst has a crystal size of up to about 1.2 μm (e.g., up to about 1.1 μm, up to about 1.0 μm, about ≤0.2 μm by about ≤0.6 μm or about 0.1 μm by about 0.5 μm).

In any aspect or embodiment described herein, the at least one protonated zeolite isomerization catalyst has a clay binder or an alumina binder.

In any aspect or embodiment described herein, the at least one protonated zeolite isomerization catalyst is present in an amount of less than about 5.0 wt % (e.g., less than about 4.0wt %, less than about 3.0 wt %, less than about 2.5 wt %, less than about 2.0 wt %, less than about 0.6 wt %, less than or equal to about 1.5 wt %, about 0.5 to about 5.0 wt %, about 0.5 to about 3.0 wt %, about 0.5 wt % to about 2.5 wt %, about 0.5 wt % to about 2.0 wt %, about 0.5 wt % to about 1.5 wt %, about 2.0 wt % to 5.0 wt %, about 2.0 wt % to 4.5 wt %, about 2.0 wt % to 4.0 wt %, or about 2.5 wt % to 4.0 wt %) of the isomerization reaction. For example, in any aspect or embodiment described herein, the at least one protonated zeolite isomerization catalyst is present in an amount of less than about 5.0 wt %, less than about 4.5 wt %, less than about 4.0 wt %, less than about 3.5 wt %, less than about 3.0 wt %, less than about 2.5 wt %, less than about 2.0 wt %, less than about 1.5 wt %, less than about 1.0 wt %, about 0.5 wt % to about 5.0 wt %, about 0.5 wt % to about 4.5 wt %, about 0.5 wt % to about 4.0 wt %, about 0.5 wt % to about 3.5 wt %, about 0.5 wt % to about 3.0 wt %, about 0.5 wt % to about 2.5 wt %, about 0.5 wt % to about 2.0 wt %, about 0.5 wt % to about 1.5 wt %, about 1.0 wt % to about 5.0 wt %, about 1.0 wt % to about 4.5 wt %, about 1.0 wt % to about 4.0 wt %, about 1.0 wt % to about 3.5 wt %, about 1.0 wt % to about 3.0 wt %, about 1.0 wt % to about 2.5 wt %, about 1.0 wt % to about 2.0 wt %, about 2.0 wt % to about 5.0 wt %, about 2.0 wt % to about 4.5 wt %, about 2.0 wt % to about 4.0 wt %, about 2.0 wt % to about 3.5 wt %, about 2.0 wt % to about 3.0 wt %, about 3.0 wt % to about 5.0 wt %, about 3.0 wt % to about 4.5 wt %, about 3.0 wt % to about 4.0 wt %, about 3.5 wt % to about 5.0 wt %, about 3.5 wt % to about 4.5 wt %, or about 4.0 wt % to about 5.0 wt %.

In any aspect or embodiment described herein, the at least one protonated zeolite isomerization catalyst is in a pellet form. In any aspect or embodiment described herein, the method further comprises, consists essentially of, or consists of, regenerating the at least one protonated zeolite isomerization catalyst (e.g., the at least one protonated zeolite isomerization catalyst is in a pellet form) by heating (e.g., heating to a temperature of about 100° C. to about 300° C. for at least 2 hours, such as about 2 hours to about 5 hours) the at least one protonated zeolite isomerization catalyst. In any aspect or embodiment described herein, the at least one protonated zeolite isomerization catalyst (e.g., the at least one protonated zeolite isomerization catalyst is in a pellet form) is located in a fixed bed of a reactor, and the method optionally further comprises, consists essentially of, or consists of, removing the at least one protonated zeolite isomerization catalyst from the fixed bed prior to heating.

In any aspect or embodiment described herein, the at least one protonated zeolite isomerization catalyst is in a pellet form, and the method further comprises, consists essentially of, or consists of, regenerating the at least one protonated zeolite isomerization catalyst (e.g., the at least one protonated zeolite isomerization catalyst is in a pellet form) by heating (e.g., heating to a temperature of about 100° C. to about 300° C. for at least 2 hours, such as about 2 hours to about 5 hours) the at least one protonated zeolite isomerization catalyst. In any aspect or embodiment described herein, the at least one protonated zeolite isomerization catalyst (e.g., the at least one protonated zeolite isomerization catalyst is in a pellet form) is located in a fixed bed of a reactor, and the method optionally further comprises, consists essentially of, or consists of, removing the at least one protonated zeolite isomerization catalyst from the fixed bed prior to heating.

In any aspect or embodiment described herein, the at least one protonated zeolite isomerization catalyst is in a pellet form, and the method further comprises, consists essentially of, or consists of, regenerating the at least one protonated zeolite isomerization catalyst (e.g., the at least one protonated zeolite isomerization catalyst is in a pellet form) by heating (e.g., heating to a temperature of about 300° C. to about 500° C. for at least 2 hours, such as about 2 hours to about 5 hours) the at least one protonated zeolite isomerization catalyst. In any aspect or embodiment described herein, the at least one protonated zeolite isomerization catalyst (e.g., the at least one protonated zeolite isomerization catalyst is in a pellet form) is located in a fixed bed of a reactor, and the method optionally further comprises, consists essentially of, or consists of, removing the at least one protonated zeolite isomerization catalyst from the fixed bed prior to heating.

In any aspect or embodiment described herein, recovering or isolating the at least one protonated zeolite isomerization catalyst comprises filtering the branched unsaturated fatty acids or alkyl esters to obtain a filtrate comprising filtered branched unsaturated fatty acids or alkyl esters and a particulate comprising the at least one protonated zeolite isomerization catalyst, and optionally, regenerating the at least one protonated zeolite isomerization catalyst through at least one of an acid treatment (e.g., an acid treatment comprising an acid solution and a solvent, such as a polar solvent or a non-polar solvent), a heat treatment (e.g., 100° C. to about 500° C. for about 2 hours to about 5 hours), or a combination thereof, of the particulate.

### Oleic Acid Composition

In any aspect or embodiment described herein, the oleic acid composition comprises at least about 30%, at least about 35%, at least about 40%, at least about 45%, at least about 50%, at least about 55%, or at least about 60% of at least one of oleic acid, linoleic acid, or a combination thereof. For example, in any aspect or embodiment described herein, the oleic acid composition comprises at least about 30%, at least about 35%, at least about 40%, at least about 45%, at least about 50%, at least about 55%, or at least about 60% oleic acid.

In any aspect or embodiment described herein, the oleic acid composition comprises less than about 90% of at least one of oleic acid, linoleic acid, or a combination thereof (e.g., less than about 90% oleic acid). For example, in any aspect or embodiment described herein, the oleic acid composition comprises less than about 90%, less than about 85%, less than about 80%, less than about 75%, less than about 70%, less than about 65%, less than about 60%, less than about 55%, less than about 50%, less than about 45%, less than about 40%, less than about 35%, about 30% to less than about 90%, about 35% to less than about 90%, about 40% to less than about 90%, about 45% to less than about 90%, about 50% to less than about 90%, about 55% to less than about 90%, about 60% to less than about 90%, about 65% to less than about 90%, about 70% to less than about 90%, about 75% to less than about 90%, about 80% to less than about 90%, about 85% to less than about 90%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 30% to about 70%, about 35% to about 70%, about 40% to about 70%, about 45% to about 70%, about 50% to about 70%, about 55% to about 70%, about 60% to about 70%, about 65% to about 70%, about 30% to about 65%, about 35% to about 65%, about 40% to about 65%, about 45% to about 65%, about 50% to about 65%, about 55% to about 65%, about 60% to about 65%, about 30% to about 60%, about 35% to about 60%, about 40% to about 60%, about 45% to about 60%, about 50% to about 60%, about 55% to about 60%, about 30% to about 55%, about 35% to about 55%, about 40% to about 55%, about 45% to about 55%, about 50% to about 55%, about 30% to about 50%, about 35% to about 50%, about 40% to about 50%, about 45% to about 50%, about 30% to about 45%, about 35% to about 45%, about 40% to about 45%, about 30% to about 40%, about 35% to about 40%, or about 30% to about 35% of at least one of oleic acid, linoleic acid, or a combination thereof (e.g., a mixture of oleic acid and linoleic acid). By way of further example, in any aspect or embodiment described herein, the oleic acid composition comprises less than about 90%, less than about 85%, less than about 80%, less than about 75%, less than about 70%, less than about 65%, less than about 60%, less than about 55%, less than about 50%, less than about 45%, less than about 40%, less than about 35%, about 30% to less than about 90%, about 35% to less than about 90%, about 40% to less than about 90%, about 45% to less than about 90%, about 50% to less than about 90%, about 55% to less than about 90%, about 60% to less than about 90%, about 65% to less than about 90%, about 70% to less than about 90%, about 75% to less than about 90%, about 80% to less than about 90%, about 85% to less than about 90%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 30% to about 70%, about 35% to about 70%, about 40% to about 70%, about 45% to about 70%, about 50% to about 70%, about 55% to about 70%, about 60% to about 70%, about 65% to about 70%, about 30% to about 65%, about 35% to about 65%, about 40% to about 65%, about 45% to about 65%, about 50% to about 65%, about 55% to about 65%, about 60% to about 65%, about 30% to about 60%, about 35% to about 60%, about 40% to about 60%, about 45% to about 60%, about 50% to about 60%, about 55% to about 60%, about 30% to about 55%, about 35% to about 55%, about 40% to about 55%, about 45% to about 55%, about 50% to about 55%, about 30% to about 50%, about 35% to about 50%, about 40% to about 50%, about 45% to about 50%, about 30% to about 45%, about 35% to about 45%, about 40% to about 45%, about 30% to about 40%, about 35% to about 40%, or about 30% to about 35% oleic acid.

In any aspect or embodiment described herein, the oleic acid composition comprises or is AltaVeg™ D 600 (Ingevity, Corp.; North Charleston, South Carolina). In any aspect or embodiment described herein, the oleic acid composition comprises oleic acid (e.g., up to about 90%, about 65% to about 85%, about 65% to about 80%, or about 73.39%), and at least one or more of linoleic acid (e.g., about 10% to about 20% or about 16.26%), stearic acid (e.g., up to about 5% or about 1.84%), palmitic acid (e.g., up to about 5% or 1.88%), other C18-1 fatty acids (e.g., fatty acids other than oleic acid about 2% to about 6% or about 4%), or a combination thereof. In any aspect or embodiment described herein, the oleic acid composition comprises or is Altapyne® L-5 (Ingevity, Corp.; North Charleston, South Carolina). In any aspect or embodiment described herein, the oleic acid composition comprises, consists essentially of, or consists of, tall oil fatty acids (TOFA) (e.g., TOFA with up to about 6% or about 5%). For example, in any aspect or embodiment described herein, the oleic acid composition comprises oleic acid (e.g., about 30% to about 40%), linoleic acid (e.g., about 30% to about 40%), and optionally, rosin (e.g., up to about 6% or about 5%).

In any aspect or embodiment described herein, the oleic acid composition is present in an amount of greater than or equal to about 85.0 weight percent (wt %) (e.g., greater than or equal to about 90.0 wt %, about 85.0 weight percent (wt %) to about 95.0 wt % or about 90 wt % to about 95 wt %) of the isomerization reaction.

### Lewis Based or Oligomerization Inhibitor

In any aspect or embodiment described herein, at least one of (i) the isomerization reaction does not include a Lewis base (e.g., an oligomerization inhibitor); (ii) the isomerization reaction is performed for up to about 30 hours (e.g., up to about 26 hours, up to about 20 hours, up to about 15 hours, up to about 10 hours, up to about 6 hours, up to about 4 hours, about 1 to about 30 hours, about 1 to about 26 hours, about 1 to about 20 hours, about 1 to about 15 hours, about 1 to about 10 hours, about 1 to about 6 hours, about 1 hour to about 4 hours, about 2 hours to about 6 hours, or about 2 hours to about 4 hours); or (iii) a combination thereof.

In any aspect or embodiment described herein, the isomerization reaction does not include a Lewis base (e.g., an oligomerization inhibitor). In any aspect or embodiment described herein, when isomerization reaction does not include a Lewis base (e.g., an oligomerization inhibitor), the isomerization reaction is performed for up to about 10 hours (e.g., up to about 6 hours, up to about 4 hours, about 1 to about 10 hours, about 1 to about 6 hours, about 1 hour to about 4 hours, about 2 hours to about 6 hours, or about 2 hours to about 4 hours).

In any aspect or embodiment described herein, at least one of: the additional isomerization reaction does not include a Lewis base (e.g., an oligomerization inhibitor); the additional isomerization reaction is performed for up to about 15 hours (e.g., up to about 12 hours, up to about 10 hours, about 1 to about 15 hours, about 1 to about 12 hours, about 1 to about 10 hours, about 5 to about 15 hours, about 5 to about 12 hours, about 7 to about 11 hours, or about 8 hour to about 10 hours); or a combination thereof.

In any aspect or embodiment described herein, the additional isomerization reaction does not include a Lewis base (e.g., an oligomerization inhibitor). In any aspect or embodiment described herein, when the additional isomerization reaction does not include an oligomerization inhibitor, the additional isomerization reaction is performed for up to about 15 hours (e.g., up to about 12 hours, up to about 10 hours, about 1 to about 15 hours, about 1 to about 12 hours, about 1 to about 10 hours, about 5 to about 15 hours, about 5 to about 12 hours, about 7 to about 11 hours, or about 8 hour to about 10 hours).

In any aspect or embodiment described herein, the isomerization reaction further comprises, consists essentially of, or consists of, a Lewis base (e.g., an oligomerization inhibitor). For example, in any aspect or embodiment described herein, the Lewis base or the oligomerization inhibitor is present in an amount of up to about 1.5 wt % (e.g., ≤about 1.5 wt %, ≤about 1.25 wt %, ≤about 1.0 wt %, ≤about 0.75 wt %, ≤about 0.5 wt %, ≤about 0.25 wt %, about 0.01 to about 1.5 wt %, about 0.01 wt % to about 1.0 wt %, about 0.01 wt % to about 0.5 wt %, about 0.01 wt % to about 0.25 wt %, about 0.1 to about 1.5 wt %, about 0.1 wt % to about 1.0 wt %, about 0.1 wt % to about 0.5 wt %, about 0.2 to about 1.5 wt %, about 0.2 wt % to about 1.0 wt %, about 0.2 wt % to about 0.5 wt %, about 0.5 to about 1.5 wt %, about 0.5 wt % to about 1.0 wt %, about 0.5 wt % to about 1.0 wt %, or about 0.5 wt % to about 0.75 wt %) of the isomerization reaction. In any aspect or embodiment described herein, when the isomerization reaction further comprises, consists essentially of, or consists of, a Lewis base (e.g., an oligomerization inhibitor), the isomerization reaction is performed for about 24 to about 48 hours (e.g., about 24 to about 42 hours, about 24 to about 36 hours, about 1 hour to about 4 hours, about 2 hours to about 6 hours, or about 2 hours to about 4 hours). For example, in any aspect or embodiment described herein, when the isomerization reaction further comprises, consists essentially of, or consists of, a Lewis base (e.g., an oligomerization inhibitor), the isomerization reaction is performed for about 24 to about 48 hours, about 28 to about 48 hours, about 32 to about 48 hours, about 36 to about 48 hours, about 40 to about 48 hours, about 44 to about 48 hours, about 24 to about 44 hours, about 28 to about 44 hours, about 32 to about 44 hours, about 36 to about 44 hours, about 40 to about 44 hours, about 24 to about 40 hours, about 28 to about 40 hours, about 32 to about 40 hours, about 36 to about 40 hours, about 24 to about 36 hours, about 28 to about 36 hours, about 32 to about 36 hours, about 24 to about 32 hours, about 28 to about 32 hours, or about 24 to about 28 hours.

In any aspect or embodiment described herein, the Lewis base or the oligomerization inhibitor includes or is at least one of an amine, phosphine, triarylphosphine, quaternary ammonium salt, dialkylarylphosphine, trialkylphosphine, or a combination thereof.

In any aspect or embodiment described herein, the Lewis base or the oligomerization inhibitor includes or is at least one amine of dimethylamine, trimethylamine, diethylamine, trimethylamine, diisopropylamine, triisopropylamine, triphenylamine diphenylamine, or a combination thereof. In any aspect or embodiment described herein, the Lewis base or the oligomerization inhibitor includes or is at least one phosphine of methylphosphine, butylphosphine, dibutylphosphine, tributylphosphine, phenylphosphine, diphenylphosphine, or a combination thereof. In any aspect or embodiment described herein, the Lewis base or the oligomerization inhibitor includes or is at least one triarylphosphine of triphenylphosphine, tri-p-tolylphosphine, tri(o-tolyl)phosphine, tri-m-tolylphosphine, trixylyl-phosphine, tris(p-ethylphenyl)phosphine, tris(p-methoxyphenyl)phosphine, tris(4-fluorophenyl)phosphine, tris(4-methoxyphenyl)phosphine, tris(dimethylamino)phosphine, tris(trimethylsilyl)phosphine, triisopropylphosphine, or a combination thereof. In any aspect or embodiment described herein, the Lewis base or the oligomerization inhibitor includes or is a tetraalkyl quaternary ammonium compound (e.g., dialkyl dimethyl ammonium chloride or didecyl dimethyl ammonium chloride), an alkyltrimethyl quaternary ammonium compound (e.g., alkyl dimethyl benzyl ammonium chloride), a dialkyldimethyl quaternary ammonium compound (e.g., dialkyl dimethyl ammonium chloride), a pyridinium quaternary ammonium compound, a benzylalkyldimethyl quaternary ammonium compound, or a combination thereof. In any aspect or embodiment described herein, the Lewis base or the oligomerization inhibitor includes or is at least one quaternary ammonium salt of benzalkonium chloride, benzethonium chloride, methylbenzethonium chloride, cetalkonium chloride, cetylpyridinium chloride, cetrimonium, cetrimide, dofanium chloride, tetraethylammonium bromide, tetrabutylammonium bromide didecyldimethylammonium chloride and domiphen bromide, or a combination thereof. In any aspect or embodiment described herein, the Lewis base or the oligomerization inhibitor includes or is at least one dialkylarylphosphine of di-n-butylphenylphosphine, dicyclohexylphenylphosphine, or a combination thereof. In any aspect or embodiment described herein, the Lewis base or the oligomerization inhibitor includes or is at least one trialkylphosphine of tri-n-butylphosphine, tricyclohexylphosphine, tri-n-octylphosphine, trimethyphosphine, triethylphosphine, triisopropylphosphine, tricyclopentylphosphine, or a combination thereof.

In any aspect or embodiment described herein, the Lewis base or the oligomerization inhibitor includes or is triphenylphosphine, tetrabutylammonium bromide, or a mixture thereof.

### Distilling

In any aspect or embodiment described herein, the method further comprises, consists essentially of, or consists of, distilling the branched unsaturated fatty acids or alkyl esters (e.g., branched unsaturated fatty acids or alkyl esters from the isomerization reaction, the additional isomerization reaction, or the hydrogenation reaction).

In any aspect or embodiment described herein, distilling the branched unsaturated fatty acids or alkyl esters includes at least one of: (i) isolating the branched unsaturated fatty acids or alkyl esters; (ii) isolating the zeolite isomerization catalyst; (iii) isolating at least one of dimer fatty acids, trimer fatty acids, or a combination thereof; or (iv) a combination thereof.

In any aspect or embodiment described herein, distilling is wiped film evaporator vacuum distillation. For example, in any aspect or embodiment described herein, distilling is wiped film evaporator vacuum distillation, and at least one of: (i) a still body jacket of the wiped film evaporator is heated to a temperature of about 220° C. to about 260° C. (e.g., about 240° C.) with vacuum (e.g., about 4 mmHg to about 8 mmHg or about 6 mmHg); (ii) a condenser of the wiped film evaporator is set to a temperature of about 50° C. to about 70° C. (e.g., about 50° C. to about 70° C., about 50° C. to about 60° C., about 60° C. to about 70° C., about 55° C. to about 65° C., or about 60° C.); (iii) a cold trap of the wiped film evaporator is set to a temperature of about 3° C. to about 10° C. (e.g., about 3° C., about 4° C., about 5° C., about 6° C., about 7° C., about 8° C., about 9° C., about 10° C., or any values in between, or a range from any combination of the values); or (iv) a combination thereof.

### Hydrogenating

In any aspect or embodiment described herein, the hydrogenation catalyst does not include a palladium-based catalyst.

In any aspect or embodiment described herein, the further comprises, consists essentially of, or consists of, hydrogenating the branched unsaturated fatty acids or alkyl esters thereof (e.g., the branched unsaturated fatty acids or alkyl esters isolated through distilling or the filtered branched unsaturated fatty acids or alkyl esters) in the presence of a hydrogenation catalyst to produce branched saturated fatty acids or alkyl esters thereof.

In any aspect or embodiment described herein, hydrogenating the branched unsaturated fatty acids or alkyl esters thereof comprises at least one of: (i) heating a reaction mixture to about 200° C. to about 230° C. (e.g., about 200° C. to about 230° C., about 200° C. to about 220° C., about 200° C. to about 210° C., about 210° C. to about 230° C., about 210° C. to about 220° C., about 220° C. to about 230° C., or about 215° C.); (ii) pressurizing a reaction mixture (e.g., pressurizing the reaction mixture with hydrogen) to about 90 pounds per square inch to about 110 pounds per square inch (e.g., about 90 to about 110, about 90 to about 100, about 100 to about 110 pounds per square inch); (iii) reacting a reaction mixture until the oleic acid content is below about 2% (e.g., below about 1.9%, below about 1.8%, below about 1.7%, below about 1.6%, below about 1.5%, below about 1.4%, below about 1.3%, below about 1.2%, below about 1.1%, below about 1.0%, below about 0.9%, below about 0.8%, below about 0.7%, below about 0.6%, or below about 0.5%); (iv) reacting a reaction mixture for at least about 5 hours (e.g., about 5 hours to about 18 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or any values in between, or a range from any combination of the values); or (v) a combination thereof.

In any aspect or embodiment described herein, the hydrogenation catalyst comprises at least one of a spongy nickel catalyst (e.g., Raney® nickel), a supported nickel catalyst (e.g., at least one of a silica-alumina support nickel, an alumina support nickel, silica supported nickel, or a combination thereof), a polymer-supported nickel catalyst, or a combination thereof. In any aspect or embodiment described herein, the hydrogenation catalyst is present in an amount of less than or equal to about 3 wt % (e.g., less than or equal to about 2 wt % or less than or equal to about 1 wt %) of the reaction mixture. In any aspect or embodiment described herein, the branched unsaturated fatty acids or alkyl esters thereof is present in an amount of at least about 97 wt % (e.g., at least about 98 wt % or at least about 99 wt %) of the reaction mixture.

In any aspect or embodiment described herein, hydrogenating the branched unsaturated fatty acids or alkyl esters thereof comprises preparing a reaction mixture comprising a hydrogenation catalyst and the branched unsaturated fatty acids or alkyl esters thereof (e.g., the filtered branched unsaturated fatty acids or alkyl esters or the branched unsaturated fatty acids or alkyl esters isolated through distilling).

### Compositions

An aspect of the present disclosure provides a composition produced according to the method of the present disclosure. For example, in any aspect or embodiment described herein, the composition (e.g., a composition produced from the isomerization reaction or the isomerization reaction and the additional isomerization reaction) comprises, consists essentially of, or consists of: branched unsaturated fatty acids or alkyl esters monomers in an amount of about 30.0 wt % to about 90.0 wt %, based on the total weight of the composition; and branched unsaturated fatty acid or alkyl ester oligomers (e.g., dimers) in an amount of about 10.0 wt % to about 30.0 wt %, based on the total weight of the composition.

In any aspect or embodiment described herein, the composition (e.g., a composition produced from hydrogenating (a) the isomerization reaction product or (b) the additional isomerization reaction product) comprises, consists essentially of, or consists of: branched unsaturated fatty acids or alkyl esters monomers in an amount of about 30.0 wt % to about 90.0 wt %, based on the total weight of the composition; and branched unsaturated fatty acid or alkyl ester oligomers (e.g., dimers) in an amount of about 10.0 wt % to about 30.0 wt %, based on the total weight of the composition.

### EXAMPLES

### Analytical Methods

Percent Water. Water content (%) was examined via American Society for Testing and Materials (ASTM) D630 Standard Test Method for Determination of Water in Petroleum Products, Lubricating Oils, and Additives by Coulometric Karl Fischer Titration. This test method covers the direct determination of entrained water in petroleum products and hydrocarbons using automated instrumentation. This test method also covers the indirect analysis of water thermally removed from samples and swept with dry inert gas into the Karl Fischer titration cell. Mercaptan, sulfide (S— or H₂S), sulfur, and other compounds are known to interfere with this test method (see Section 6). The precision statement of this method covers the nominal range of 20 mg/kg to 25,000 mg/kg for Procedure A, 30 mg/kg to 2,100 mg/kg for Procedure B, and 20 mg/kg to 360 mg/kg for Procedure C.

This test method is intended for use with commercially available coulometric Karl Fischer reagents and for the determination of water in additives, lube oils, base oils, automatic transmission fluids, hydrocarbon solvents, and other petroleum products. By proper choice of the sample size, this test method may be used for the determination of water from mg/kg to percent level concentrations.

Acid Number. Acid number (mgKOH/g) was examined via American Society for Testing and Materials (ASTM) D664-18e2 Standard Test Method for Acid Number of Petroleum Products by Potentiometric Titration. For petroleum products and lubricants soluble or nearly soluble in mixtures of toluene and propan-2-ol. It is applicable for the determination of acids whose dissociation constants in water are larger than 10⁻⁹; extremely weak acids whose dissociation constants are smaller than 10⁻⁹ do not interfere. Salts react if their hydrolysis constants are larger than 10⁻⁹. The range of acid numbers included in the precision statement is 0.1 mg/g KOH to 150 mg/g KOH.

Saponification Value. Saponification value (mg KOH/g) was examined via American Society for Testing and Materials (ASTM) D1962 Standard Test Method for Saponification Value of Drying Oils, Fatty Acids, and Polymerized Fatty Acids. This test method covers the determination of the saponification value of drying oils, bodied oils, fatty acids, and polymerized fatty acids.

Iodine Value. Iodine value (gL/100g≤8.00) was examined via American Society for Testing and Materials (ASTM) D5768-02 Standard Test Method for Determination of Iodine Value of Tall Oil Fatty Acids. This test method covers the Wijs procedure for determination of unsaturation (iodine value) of tall oil fatty acids. Iodine value is a measure of the unsaturation of oils and fatty acids and is expressed in terms of the number of centigrams of iodine per gram of sample (weight percent of absorbed iodine).

Cloud Point. Cloud point (° C.≤8) was examined via American Society for Testing and Materials (ASTM) D2500-17a Standard Test Method for Cloud Point of Petroleum Products and Liquid Fuels. This test method covers only petroleum products and biodiesel fuels that are transparent in layers 40 millimeter in thickness, and with a cloud point below 49° C.

Fatty Acid Composition. The fatty acid composition (chain length, saturated/unsaturated, linear/branched) was determined using gas chromatography, International Organization for Standardization (ISO) 5508:1990 (E) Animal and vegetable fats and oils—Analysis by gas chromatography of methyl esters of fatty acids. Gas chromatography (GC) was used to determine the percentage by weight (wt %) composition of products in the crude isomerized reaction mixtures after hydrogenation and methylation. Gas chromatography (GC) was equipped with a capillary inlet injector (on column mode) and flame ionization detection (FID). The capillary column used was an HP Agilent DB5-HT column (30 m×0.1 mm×0.32 um) (Santa Clara, California, United States of America) attached to an Alltech Co. (State College, Pennsylvania, United States of America) deactivated fused-silica guard column (3 m×0.32 um). Helium was the carrier gas set at constant flow of 6 mL/minute. The detector temperature was set at 390° C. The oven temperature profile used was: initial temperature 50° C., hold for 1 minute; ramp at 15° C./minute to 160° C.; ramp at 78° C./minute to 230° C.; ramp at 30° C./minute to 380° C. hold for 10 minutes.

### Example 1: Isomerization of Comparative Example A

Four-hundred grams (g) of technical grade, 90% oleic acid (Sigma-Aldrich®; Saint Louis, Missouri) was charged to a 600 ml Parr reactor, then triphenylphosphine (TPP; 1.5 g; Aldrich Chemical, Milwaukee, Wisconsin), water (8 g), and H-Ferrierite (20 g), which was prepared from 500° C., 5 hour treatment of HSZ®-720NHA (TOSOH Corporation; Grove City, Ohio). The reaction mixture was pressurized at 120 pounds per square inch (psi), and then heated to 260° C. The pressure rose to a pressure of 281 pounds per square inch after 6 hours. After 6 hours, the conversion rate based on oleic acid was 81%. After overnight heating, the final pressure was 315 pounds per square inch. The reaction was stopped after 22 hours. The final conversion rate based on oleic acid was 94.8% and dimer formation was 6.1%.

### Example 2: Isomerization of Comparative Example B

Four-hundred grams of technical grade, 90% oleic acid (Sigma-Aldrich®; Saint Louis, Missouri) was charged to a 600 ml Parr reactor, then triphenylphosphine (TPP; 1.5 g; Aldrich Chemical, Milwaukee, Wisconsin), water (8 g), and H-Ferrierite (20 g), which was prepared from HCl treatment using HSZ®-720KOA (TOSOH Corporation; Grove City, Ohio). The reaction mixture was pressurized at 120 pounds per square inch, then heated to 260° C. The pressure rose to 275 pounds per square inch after 6 hours. After 6 hours, the conversion rate based on oleic acid was 79%. After overnight heating, the final pressure was 289 pounds per square inch. The reaction was stopped after 22 hours. Conversion rate based on oleic acid was 92% and dimer formation was 6.3%.

### Example 3: Isomerization of Example 1

Three-hundred-sixty-five grams of technical grade, 90% oleic acid (Sigma-Aldrich®; Saint Louis, Missouri) was charged to a 600 ml Parr reactor and 14.6 g HSZ®-722HOA (TOSOH Corporation; Grove City, Ohio) was used as isomerization catalyst. Triphenylphosphine (TPP; 1.2 g; Aldrich Chemical, Milwaukee, Wisconsin) and water (7.5 g) was then added and reactor pressurized under nitrogen to 100 pounds per square inch and heated to 260° C. for 24 hours. Final pressure was 350 pounds per square inch (psi). Conversion rate based on oleic acid was 96% and dimer formation was 5.4%.

### Example 4. Isomerization of Example 2

Three-hundred-sixty-five grams of technical grade, 90% oleic acid (Sigma-Aldrich®; Saint Louis, Missouri) was charged to a 600 ml Parr reactor and 14.6 g HSZ®-720NHA (TOSOH Corporation; Grove City, Ohio) was used as isomerization catalyst. Triphenylphosphine (TPP; 1.2 g; Aldrich Chemical, Milwaukee, Wisconsin) and water (7.5 g) was then added and reactor pressurized under nitrogen to 100 pounds per square inch and heated to 260° C. for 24 hours. Final pressure was 350 pounds per square inch (psi). Conversion rate based on oleic acid was 95% and dimer formation was 5.4%.

### Example 5: Isomerization of Example 3

Three-hundred-sixty-five of grams AltaVeg™ D 600 (Ingevity, Corp.; North Charleston, South Carolina) was charged to a 600 ml Parr reactor and 14.6 g HSZ®-722HOA (TOSOH Corporation; Grove City, Ohio) was used as isomerization catalyst. Triphenylphosphine (TPP; 1.2 g; Aldrich Chemical, Milwaukee, Wisconsin) and water (7.5 g) was then added and reactor pressurized under nitrogen to 70 pounds per square inch and heated to 260° C. for 26 hour. Final pressure was 180 pounds per square inch. Conversion rate based on oleic acid was 96% and dimer formation was 24%.

### Example 6: Isomerization of Example 4

Three-hundred-sixty-five grams of AltaVeg™ D 600 (Ingevity, Corp.; North Charleston, South Carolina) was charged to a 600 ml Parr reactor and 14.6 g HSZ®-723HOA (TOSOH Corporation; Grove City, Ohio) was used as isomerization catalyst. Triphenylphosphine (TPP; 1.2 g; Aldrich Chemical, Milwaukee, Wisconsin) and water (7.5 g) was then added and reactor pressurized under nitrogen to 40 pounds per square inch and heated to 260° C. for 30 hours. Final pressure was 110 pounds per square inch. Conversion rate based on oleic acid was 96% and dimer formation was 21%.

### Example 7: Isomerization of Example 5

Three-hundred-sixty-five grams of AltaVeg™ D 600 (Ingevity, Corp.; North Charleston, South Carolina) was charged to a 600 ml Parr reactor and 14.6 g HSZ®-723HOA (TOSOH Corporation; Grove City, Ohio) was used as isomerization catalyst. Tetrabutylammonium bromide (TBAB; 1.2 g; Aldrich Chemical, Milwaukee, Wisconsin) and 5 g water was then added and reactor pressurized under nitrogen to 40 pounds per square inch and heated to 260° C. for 30 hours. Final pressure was 110 pounds per square inch. Conversion rate based on oleic acid was 97% and dimer formation was 16%.

### Example 8: Isomerization of Example 6

Three-hundred-sixty-five grams of AltaVeg™ D 600 (Ingevity, Corp.; North Charleston, South Carolina) was charged to a 600 ml Parr reactor and 14.6 g HSZ®-723HOA (TOSOH Corporation; Grove City, Ohio) was used as isomerization catalyst. No additive was added and 5 g water was then added and reactor pressurized under nitrogen to 40 pounds per square inch and heated to 260° C. for 6 hours. Final pressure was in the range of 220 pounds per square inch. Conversion rate based on oleic acid was 97% and dimer formation was 31%.

### Example 9: Isomerization of Example 7

Three-hundred-sixty-five grams of AltaVeg™ D 600 (Ingevity, Corp.; North Charleston, South Carolina) was charged to a 600 ml Parr reactor and 14.6 g HSZ®-723HOA (TOSOH Corporation; Grove City, Ohio) was used as isomerization catalyst. No additive was added and 5 g water was then added and reactor pressurized under nitrogen to 40 pounds per square inch and heated to 260° C. for 30 hours. Final pressure was in the range of 220 pounds per square inch. Conversion rate based on oleic acid was 98% and dimer formation was 31%.

### Example 10: Isomerization of Example 8

Three-hundred-sixty-five grams of AltaVeg™ D 600 (Ingevity, Corp.; North Charleston, South Carolina) was charged to a 600 ml Parr reactor and 14.6 g HSZ®-822HOA (TOSOH Corporation; Grove City, Ohio) was used as isomerization catalyst. Triphenylphosphine (TPP; 1.2 g; Aldrich Chemical, Milwaukee, Wisconsin) and water (5 g) was then added and reactor pressurized under nitrogen to 100 pounds per square inch and heated to 260° C. for 24 hours. Final pressure was 400 pounds per square inch. Conversion rate based on oleic acid was 98% and dimer formation was 18%.

### Example 11: Isomerization of Example 9

Three-hundred-sixty-five grams of AltaVeg™ D 600 (Ingevity, Corp.; North Charleston, South Carolina) was charged to a 600 ml Parr reactor and 14.6 g HSZ®-822HOA (TOSOH Corporation; Grove City, Ohio) was used as isomerization catalyst. Triphenylphosphine (TPP; 1.2 g; Aldrich Chemical, Milwaukee, Wisconsin) and water (5 g) was then added and reactor pressurized under nitrogen to 40 pounds per square inch and heated to 260° C. for 24 hours. Final pressure was 110 pounds per square inch. Conversion rate based on oleic acid was 98% and dimer formation was 18%.

TABLE 1
Summary of the isomerization examples

| | | Zeolite | | Reaction | Iso-oleic | Dimer | Conversion |
|---|---|---|---|---|---|---|---|
| Example | FFA | (charge) | Additive | Conditions | (%) | (%) | (%) |
| A | 90% oleic | HSZ ®- | TPP | 22 hours/315 | 65 | 6.1 | 95 |
| | | 720KOA | | psi, 260° C. | | | |
| | | (4%) | | | | | |
| B | 90% oleic | HSZ ®- | TPP | 22 hours/289 | 74 | 6.3 | 92 |
| | | 720NHA | | psi, 260° C. | | | |
| | | (4%) | | | | | |
| 1 | 90% oleic | HSZ ®- | TPP | 24 hours/350 | 75 | 5.4 | 96 |
| | | 722HOA | | psi, 260° C. | | | |
| | | (4%) | | | | | |
| 2 | 90% oleic | HSZ ®- | TPP | 24 hours/350 | 75 | 5.4 | 96 |
| | | 722HOA | | psi, 260° C. | | | |
| | | (4%) | | | | | |
| 3 | 70% oleic | HSZ ®- | TPP | 26 hours/180 | 71 | 24 | 96 |
| | | 722HOA | | psi, 260° C. | | | |
| | | (4%) | | | | | |
| 4 | 70% oleic | HSZ ®- | TPP | 30 hours/110 | 71 | 21 | 96 |
| | | 722HOA | | psi, 260° C. | | | |
| | | (4%) | | | | | |
| 5 | 70% oleic | HSZ ®- | TBAB | 30 hours/110 | 74 | 16 | 97 |
| | | 723HOA | | psi, 260° C. | | | |
| | | (4%) | | | | | |
| 6 | 70% oleic | HSZ ®- | None | 6 hours/ 220 | 72 | 31 | 97 |
| | | 723HOA | | psi, 260° C. | | | |
| | | (4%) | | | | | |
| 7 | 70% oleic | HSZ ®- | None | 6 hours/ 220 | 72 | 31 | 97 |
| | | 723HOA | | psi, 260° C. | | | |
| | | (4%) | | | | | |
| 8 | 70% oleic | HSZ ®- | TPP | 24 hours/400 | 66 | 18 | 98 |
| | | 822HOA | | psi, 260° C. | | | |
| | | (4%) | | | | | |
| 9 | 70% oleic | HSZ ®- | TPP | 24 hours/400 | 66 | 18 | 98 |
| | | 822HOA | | psi, 260° C. | | | |
| | | (4%) | | | | | |

### Example 12: Isomerization of Example 10

Two-hundred-five grams of Alta Veg™ D 600 (Ingevity, Corp.; North Charleston, South Carolina) was charged to a 600 ml Parr reactor and 12 g HSZ®-723HOD1A pellet (TOSOH Corporation; Grove City, Ohio) was used as isomerization catalyst. Water (4.6 g) was then added, and the reactor was pressurized to 40 pounds per square inch under nitrogen and heated to 260° C. for 6 hours. Final pressure was 120 pounds per square inch. Conversion rate based on oleic acid was 95% and dimer formation was 27%.

### Example 13: Isomerization of Example 11

One-hundred-eighty grams of AltaVeg™ D 600 (Ingevity, Corp.; North Charleston, South Carolina) was charged to the 600 ml Parr reactor containing the same HSZ®-723HOD1A pellets (TOSOH Corporation; Grove City, Ohio) used in Example 11, as isomerization catalyst. Water (3.0 g) was then added, and the reactor was pressurized to 40 pounds per square inch under nitrogen and heated to 260° C. for 6 hours. Final pressure was 120 pounds per square inch. Conversion rate based on oleic acid was 91% and dimer formation was 24%.

### Example 14: Isomerization of Example 12

The spent HSZ®-723HOD1A pellets (TOSOH Corporation; Grove City, Ohio) from Example 12 were rinsed with acetone and heated in an oven at 500° C. for 3 hours.

One-hundred-ninety-eight grams of AltaVeg™ D 600 (Ingevity, Corp.; North Charleston, South Carolina) was charged to the 600 ml Parr and 9.8 g of the washed and heated HSZ®-723HOD1A pellets used as isomerization catalyst. Water (4.0 g) was then added, and the reactor was pressurized to 50 pounds per square inch under nitrogen and heated to 265° C. for 6 hours. Final pressure was 120 pounds per square inch. Conversion rate based on oleic acid was 88% and dimer formation was 23%.

### Example 15: Isomerization of Example 13

Two-hundred-twenty-five grams of AltaVeg™ D 600 (Ingevity, Corp.; North Charleston, South Carolina) was charged to a 600 ml Parr reactor and 2.2 g HSZ®-722HOA powder (TOSOH Corporation; Grove City, Ohio) was used as isomerization catalyst. Water (4.5 g) was then added and reactor pressurized to 50 pounds per square inch under nitrogen and heated to 260° C. for 24 hours. Final pressure was 120 pounds per square inch. Conversion rate based on oleic acid was 95% and dimer formation was 35%.

### Example 16: Isomerization of Example 14

Two-hundred-twenty-five grams of AltaVeg™ D 600 (Ingevity, Corp.; North Charleston, South Carolina) was charged to a 600 ml Parr reactor and 1.0 g HSZ®-722HOA powder (TOSOH Corporation; Grove City, Ohio) was used as isomerization catalyst. Water (4.5 g) was then added and reactor pressurized to 50 pounds per square inch under nitrogen and heated to 260° C. for 30 hours. Final pressure was 120 pounds per square inch. Conversion rate based on oleic acid was 92% and dimer formation was 30%.

### Example 17: Isomerization of Example 15

Two-hundred-twenty-five grams of AltaVeg™ D 600 (Ingevity, Corp.; North Charleston, South Carolina) was charged to a 600 ml Parr reactor and 6.8 g HSZ®-722HOA powder (TOSOH Corporation; Grove City, Ohio) was used as isomerization catalyst. Water (4.5 g) was then added and reactor pressurized to 50 pounds per square inch under nitrogen and heated to 260° C. for 24 hours. Final pressure was 120 pounds per square inch. Conversion rate based on oleic acid was 99% and dimer formation was 22%.

### Example 18: Isomerization of Example 16

Two-hundred-seventeen grams of Altapyne L-5 (Ingevity, Corp.; North Charleston, South Carolina) was charged to a 600 ml Parr reactor and 6.5 g HSZ®-822HOA powder (TOSOH Corporation; Grove City, Ohio) was used as isomerization catalyst. Water (2.0 g) was then added and reactor pressurized to 40 pounds per square inch and heated to 260° C. for 24 hours. Final pressure was 120 pounds per square inch. Conversion rate based on oleic acid was 99% and dimer formation was 27%.

### Example 2. Summary of Modified Isomerization Examples

| | | Zeolite | | Reaction | Iso-oleic | Dimer | Conversion |
|---|---|---|---|---|---|---|---|
| Example | FFA | (charge) | Additive | Conditions | (%) | (%) | (%) |
| 10 | 70% oleic | HSZ ®- | None | 6 hours/120 | 80 | 27 | 95 |
| | | 723HOD1A | | psi, 260° C. | | | |
| | | (5.5%) | | | | | |
| 11 | 70% oleic | HSZ ®- | None | 6 hours/120 | 61 | 24 | 91 |
| | | 723HOD1A | | psi, 260° C. | | | |
| | | (5.5%, | | | | | |
| | | reuse) | | | | | |
| 12 | 70% oleic | HSZ ®- | None | 10 hours/120 | 68 | 23 | 88 |
| | | 723HOD1A | | psi, 260° C. | | | |
| | | (4.5%) | | | | | |
| 13 | 70% oleic | HSZ ®- | None | 24 hours/120 | 81 | 35 | 95 |
| | | 722HOA | | psi, 260° C. | | | |
| | | (1%) | | | | | |
| 14 | 70% oleic | HSZ ®- | None | 30 hours/120 | 73 | 30 | 92 |
| | | 722HOA | | psi, 260° C. | | | |
| | | (0.45%) | | | | | |
| 15 | 70% oleic | HSZ ®- | None | 24 hours/120 | 76 | 22 | 99 |
| | | 822HOA | | psi, 260° C. | | | |
| | | (3%) | | | | | |
| 16 | L-5 | HSZ ®- | None | 24 hours/120 | 73 | 27 | 99 |
| | | 822HOA | | psi, 260° C. | | | |
| | | (3%) | | | | | |

### Example 19: Two-Step Isomerization of Example 17

Step 1. 300 g AltaVeg™ D 600 (Ingevity, Corp.; North Charleston, South Carolina) was charged and 3.6 g HSZ®-722HOA zeolite powder (TOSOH Corporation; Grove City, Ohio) was used as isomerization catalyst. Water (6 g) was added and the reaction mixture was pressurized to 55 pounds per square inch under nitrogen and heated to 260° C. for 24 hours. Final pressure was 120 pounds per square inch. Conversion rate based on oleic acid was 97% and dimer formation was 27%.

Step 2. To the reaction product was added another 3.6 g HSZ®-822HOA zeolite powder (TOSOH Corporation; Grove City, Ohio). Water (6 g) was added and the reaction mixture heated to 260° C. for additional 10 hours. Conversion rate based on oleic acid was 98.3% and dimer formation was 33%.

### Example 20: Two-Step Isomerization of Example 18

Step 1. 286 g AltaVeg™ D 600 (Ingevity, Corp.; North Charleston, South Carolina) was charged and 4.26 g HSZ®-822HOA zeolite powder (TOSOH Corporation; Grove City, Ohio) was used as isomerization catalyst. Water (6 g) was added and the reaction mixture was pressurized to 45 pounds per square inch under nitrogen and heated to 260° C. for 24 hours. Final pressure was 120 pounds per square inch. Conversion rate based on oleic acid was 98.5% and dimer formation was 18%.

Step 2. To the reaction product was added another 3.6 g F-100 clay (Brenntag North America, Inc.; Reading, Pennsylvania). Water (6 g) was added and the reaction mixture heated to 260° C. for additional 10 hours. Conversion rate based on oleic acid was 100% and dimer formation was 35%.

### Example 21: Two-Step Isomerization of Example 19

Step 1. 284 g AltaVeg™ D 600 (Ingevity, Corp.; North Charleston, South Carolina) was charged and 4.26 g HSZ®-822HOA zeolite powder (TOSOH Corporation; Grove City, Ohio) was used as isomerization catalyst. Water (6 g) was added and the reaction mixture was pressurized to 55 pounds per square inch under nitrogen and heated to 260° C. for 25 hours. Final pressure was 120 pounds per square inch. Conversion rate based on oleic acid was 90% and dimer formation was 18.5%.

Step 2. To the reaction product was added another 2.6 g HSZ®-722HOA zeolite powder (TOSOH Corporation; Grove City, Ohio). Water (3 g) was added and the reaction mixture heated to 260° C. for additional 8 hours. Conversion rate based on oleic acid was 98% and dimer formation was 19.1%.

TABLE 3
Summary of two-step isomerization examples

| | | Zeolite | | Reaction | Iso-oleic | Dimer | Conversion |
|---|---|---|---|---|---|---|---|
| Example | FFA | (charge) | Additive | Conditions | (%) | (%) | (%) |
| 17 | 70% | HSZ ®- | None | 1^{st} step: 120 psi, | 79 | 33 | 98.3 |
| | oleic | 722HOA | | 260° C. for 24 | | | |
| | | (1.5%), then | | hours | | | |
| | | HSZ ®-822 | | 2^{nd} step: 10 | | | |
| | | HOA | | hours same | | | |
| | | (1.5%) | | condition | | | |
| 18 | 70% | HSZ ®- | None | 1^{st} step: 120 psi, | 75 | 35 | 100 |
| | oleic | 822HOA | | 260° C. for 24 | | | |
| | | (1.5%), then | | hours | | | |
| | | F-100 clay | | 2^{nd} step: 6 hours | | | |
| | | (1.5%) | | same condition | | | |
| 19 | 70% | HSZ ®- | None | 1^{st} step: 120 psi, | 70 | 19 | 98 |
| | oleic | 822HOA | | 260° C. for 25 | | | |
| | | (1.5%), then | | hours | | | |
| | | HSZ ®- | | 2^{nd} step: 8 hours | | | |
| | | 722HOA | | same condition | | | |
| | | (1.5%) | | | | | |

### Example 22: Wiped Film Evaporator Vacuum Distillation

The Wiped Film Evaporator (WFE) still body jacket was heated to 240° C. and internal condenser was set at 60° C. The vacuum was set at 6 mmHg. The cold trap was set at 5° C. Three-hundred-fifty grams of filtered isomerized product from Examples 1, 3 through 6, and 8 was charged to the feed flask. Once all set conditions was reached, the feed was started at about 2 drops per second. About 75/25 ratio of distillate versus residue was collected, with total of 97% mass recovery feed versus total products, with about 260 g distillate collected.

### Example 23: Hydrogenation

To a clean Parr, was charged 1% Raney®-Nickel (W.R. Grace and Co. 2800, slurry, in H₂O, active catalyst) or Nickel on silica/alumina (Sigma-Aldrich®, Saint Louis, Missouri) (neat based) under nitrogen and then charged with the crude isomerization product without distillation from Example 8 or the monomer distillate collected from Example 22 utilizing composition prepared in Example 8—i.e., 2 g Raney nickel® for 200 g distilled isomerized product. The reactor was flushed with nitrogen 3 times, then hydrogen 3 times, and then pressurized with hydrogen at 100 pounds per square inch. Then, slowly the temperature was brough up to 215° C. If a pressure drop was observed, more hydrogen was introduced to obtain a pressure of 100 pounds per square inch before the temperature reached 100° C. After the set conditions were reached, the temperature/pressure was held for 6 hours. A sample was taken for gas chromatography (GC) to measure residue oleic content. The reaction was stopped when oleic acid content was below 1%. The reaction was then cooled to 60° C.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references or publication are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range. “Combinations” is inclusive of blends, mixtures, alloys, reaction products, and the like. The terms “first”, “second”, and the like, do not denote any order, quantity, or importance, but rather are used to denote one element from another.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method of producing a branched fatty acid or alkyl esters thereof, the method comprising:
subjecting an oleic acid composition comprising at least about 30% of at least one of oleic acid, linoleic acid, or a combination thereof, to an isomerization reaction in the presence of water and at least one protonated zeolite isomerization catalyst to produce branched unsaturated fatty acids or alkyl esters thereof.

2. The method of claim 1, wherein the at least one protonated zeolite isomerization catalyst includes at least one of:
a Zeolite Socony Mobil-5 (ZSM-5) structure or a ferrierite structure;
a pore size of about 4.0 to about 9.0 angstroms;
a Brunauer, Emmett and Teller (BET) surface area of about 150 to about 400 m²/g;
a mole ratio of SiO₂ to Al₂O₃ of about 15 to about 27;
a particle size of about 3 to about 22 μm;
a crystal size of up to about 1.2 μm;
a clay binder or an alumina binder; or
a combination thereof.

3. The method of claim 1, wherein at least one of:
the oleic acid composition comprises less than about 90% of at least one of oleic acid, linoleic acid, or a combination thereof;
the oleic acid composition is present in an amount of greater than or equal to about 85.0 weight percent (wt %) of the isomerization reaction;
the water is present in an amount of no greater than about 4.0 wt % of the isomerization reaction;
the at least one protonated zeolite isomerization catalyst is present in an amount of less than about 5.0 wt % of the isomerization reaction; or a combination thereof.

4. The method of claim 1, wherein at least one of:
the isomerization reaction does not include a Lewis base;
the isomerization reaction is performed for up to about 30 hours (e.g., up to about 26 hours, up to about 20 hours, up to about 15 hours, up to about 10 hours, up to about 6 hours, up to about 4 hours, about 1 to about 30 hours, about 1 to about 26 hours, about 1 to about 20 hours, about 1 to about 15 hours, about 1 to about 10 hours, about 1 to about 6 hours, about 1 hour to about 4 hours, about 2 hours to about 6 hours, or about 2 hours to about 4 hours); or a combination thereof.

5. The method of claim 1, wherein the isomerization reaction was performed (i) at about 200° C. to about 300° C., (ii) with a final pressure of about 50 pounds per square inch to about 500 pounds per square inch, or (iii) a combination thereof of (i) and (ii).

6. The method of claim 1, wherein the isomerization reaction was performed with a final pressure of less than or equal to about 125 pounds per square inch.

7. The method of claim 1, wherein at least one of:
the method produces less than or equal to about 40% dimers;
the method provides at least about 90% conversion; or a combination thereof.

8. The method of claim 1, further comprising, consisting essentially of, or consisting of, recovering or isolating the at least one protonated zeolite isomerization catalyst, and optionally regenerating the at least one protonated zeolite isomerization catalyst.

9. The method of claim 1, wherein the at least one protonated zeolite isomerization catalyst is in a pellet form.

10. The method of claim 9, further comprising, consisting essentially of, or consisting of, regenerating the at least one protonated zeolite isomerization catalyst by heating the at least one protonated zeolite isomerization catalyst.

11. The method of claim 9, wherein the at least one protonated zeolite isomerization catalyst is located in a fixed bed of a reactor, and the method optionally further comprises, consists essentially of, or consists of, removing the at least one protonated zeolite isomerization catalyst from the fixed bed prior to heating.

12. The method of claim 8, wherein recovering or isolating the at least one protonated zeolite isomerization catalyst comprises filtering the branched unsaturated fatty acids or alkyl esters to obtain a filtrate comprising filtered branched unsaturated fatty acids or alkyl esters and a particulate comprising the at least one protonated zeolite isomerization catalyst, and optionally, regenerating the at least one protonated zeolite isomerization catalyst through at least one of an acid treatment, a heat treatment, or a combination thereof, of the particulate. 13, The method of claim 1, further comprising, consisting essentially of, or consisting of, subjecting a reaction product of the isomerization reaction or the branched unsaturated fatty acids or alkyl esters thereof to an additional isomerization reaction in the presence of water and (i) at least one protonated zeolite isomerization catalyst or (ii) a clay catalyst to produce branched unsaturated fatty acids or alkyl esters thereof.

14. The method of claim 13, wherein subjecting the reaction product of the isomerization reaction or the branched unsaturated fatty acids or alkyl esters thereof to the additional isomerization reaction comprises adding the water and (i) the at least one protonated zeolite isomerization catalyst or (ii) the clay catalyst to the isomerization reaction or the branched unsaturated fatty acids or alkyl esters thereof.

15. The method of claim 13, wherein at least one of:
the additional isomerization reaction does not include a Lewis base;
the additional isomerization reaction is performed for up to about 15 hours;
the additional isomerization reaction was performed at about 200° C. to about 300° C.;
the additional isomerization reaction was performed with a final pressure of less than or equal to about 125 pounds per square inch; or a combination thereof.

16. The method of claim 1, further comprising, consisting essentially of, or consisting of, hydrogenating the branched unsaturated fatty acids or alkyl esters thereof in the presence of a hydrogenation catalyst to produce branched saturated fatty acids or alkyl esters thereof.

17. The method of claim 16, wherein the hydrogenation catalyst does not include a palladium-based catalyst.

18. The method of claim 16, wherein hydrogenating the branched unsaturated fatty acids or alkyl esters thereof comprises at least one of:
heating a reaction mixture to about 200° C. to about 230° C.;
pressurizing a reaction mixture to about 90 pounds per square inch to about 110 pounds per square inch;
reacting a reaction mixture until the oleic acid content is below about 2%;
reacting a reaction mixture for at least about 5 hours; or
a combination thereof.

19. The method of claim 16, wherein at least one of:
the hydrogenation catalyst comprises at least one of a spongy nickel catalyst, a supported nickel catalyst, a polymer-supported nickel catalyst, or a combination thereof;
the hydrogenation catalyst is present in an amount of no greater than about 3 wt % of the reaction mixture;
the branched unsaturated fatty acids or alkyl esters thereof is present in an amount of at least about 97 wt % of the reaction mixture; or a combination thereof.

20. The method of claim 16, wherein hydrogenating the branched unsaturated fatty acids or alkyl esters thereof comprises preparing a reaction mixture comprising a hydrogenation catalyst and the branched unsaturated fatty acids or alkyl esters thereof.

21. The method of claim 1, further comprising, consisting essentially of, or consisting of, distilling the branched unsaturated fatty acids or alkyl esters.

22. The method of claim 21, wherein at least one of:
distilling is wiped film evaporator vacuum distillation;
distilling the branched unsaturated fatty acids or alkyl esters includes at least one of:
isolating the branched unsaturated fatty acids or alkyl esters;
isolating the zeolite isomerization catalyst;
isolating at least one of dimer fatty acids, trimer fatty acids, or a combination thereof; or
a combination thereof;
a combination thereof.

23. The method of claim 21, wherein distilling is wiped film evaporator vacuum distillation, and at least one of:
a still body jacket of the wiped film evaporator is heated to a temperature of about 220° C. to about 260° C. with vacuum;
a condenser of the wiped film evaporator is set to a temperature of about 50° C. to about 70° C.;
a cold trap of the wiped film evaporator is set to a temperature of about 3° C. to about 10° C.; or a combination thereof.

24. A composition comprising:
branched unsaturated fatty acids or alkyl esters monomers in an amount of about 30.0 wt % to about 90.0 wt %, based on the total weight of the composition; and
branched unsaturated fatty acid or alkyl ester oligomers in an amount of about 10.0 wt % to about 30.0 wt %, based on the total weight of the composition.
